(19) United States
(12) Patent Application Publication
(10) Pub. No.: US 2014/0296712 A1
(43) Pub. Date: Oct. 2, 2014

(54) ULTRASONIC PROBE, ULTRASONIC MEASUREMENT DEVICE, AND ULTRASONIC IMAGE DEVICE

(71) Applicant: SEIKO EPSON CORPORATION, Tokyo (JP)

(72) Inventor: Kanechika KIYOSE, Matsumoto (JP)

(73) Assignee: SEIKO EPSON CORPORATION, Tokyo (JP)

(21) Appl. No.: 14/227,387

(22) Filed: Mar. 27, 2014

(30) Foreign Application Priority Data
Mar. 29, 2013 (JP) 2013-071574

(51) Int. Cl. A61B 8/00; A61B 8/14

(52) U.S. Cl. CPC A61B 8/445; A61B 8/14
USPC 600/445; 600/459

(57) ABSTRACT

An ultrasonic probe includes an ultrasonic sensor section and a guide section. The ultrasonic sensor section has an ultrasonic transducer device. The guide section is disposed on a sensor surface on which the ultrasonic sensor section is provided. The guide section is configured and arranged such that a resistance force with respect to a target sample when the ultrasonic probe is moved in a first direction while the sensor surface contacts a surface of the target sample as the ultrasonic sensor section faces the target sample is smaller than the resistance force with respect to the target sample when the ultrasonic probe is moved in a second direction intersecting the first direction.

### CROSS-REFERENCE TO RELATED APPLICATIONS

This application claims priority to Japanese Patent Application No. 2013-071574 filed on Mar. 29, 2013. The entire disclosure of Japanese Patent Application No. 2013-071574 is hereby incorporated herein by reference.

### BACKGROUND

1. Technical Field

The present invention relates to an ultrasonic probe, an ultrasonic measurement device, and ultrasonic image device, etc.

2. Related Art

A method for obtaining a panoramic image by using an ultrasonic image device (ultrasonic diagnostic apparatus) is well known. For obtaining this panoramic image, it is necessary to perform an ultrasonic measurement while a technician moves an ultrasonic probe along a desired track by freehand. However, it is difficult to precisely move the probe along an intended track while giving a constant pressure to a body surface by consistently holding the probe in a vertical position with respect to the body surface so that there is a problem that it is difficult to obtain a precise panoramic image.

For this problem, for example, in Japanese Laid-open Patent Application No. 2007-21172, a method for guiding a movement of an ultrasonic probe by a guide rail is described. However, in this method, there are problems that it is difficult to provide a precise measurement for shapes or body types of various parts to be measured, and the device becomes complicated.

### SUMMARY

According to some aspects of the present invention, an ultrasonic probe, an ultrasonic measurement device, and an ultrasonic image device, etc. that enables to move the ultrasonic probe along a desired track can be provided.

An ultrasonic probe according to one aspect includes an ultrasonic sensor section and a guide section. The ultrasonic sensor section has an ultrasonic transducer device. The guide section is disposed on a sensor surface on which the ultrasonic sensor section is provided. The guide section is configured and arranged such that a resistance force with respect to a target sample when the ultrasonic probe is moved in a first direction while the sensor surface contacts a surface of the target sample as the ultrasonic sensor section faces the target sample is smaller than the resistance force with respect to the target sample when the ultrasonic probe is moved in a second direction intersecting the first direction.

According to one aspect of the present invention, by the guide section provided on the sensor surface of the ultrasonic probe, the movement of the ultrasonic probe in the first direction becomes easier than the movement of the second direction so that the user can precisely move the ultrasonic probe in the first direction. As a result, with the simple configuration, it is possible to easily obtain a plurality of ultrasonic images while precisely moving the ultrasonic probe along a desired track.

Also, in one aspect of the present invention, the sensor surface preferably has an elongated shape in a plan view, and a longitudinal direction of the sensor surface is preferably defined as the first direction.

Because of this, the guide section can guide the movement of the ultrasonic probe in the longitudinal direction of the sensor surface.

Further, in one aspect of the present invention, the guide section preferably has a first guide section and a second guide section with a longitudinal direction of the sensor surface is defined as the first direction, and the ultrasonic sensor section is preferably disposed between the first guide section and the second guide section.

Because of this, the first and second guide sections can guide the movement of the ultrasonic probe in the longitudinal direction of the sensor surface so that the user can precisely move the ultrasonic probe in the longitudinal direction of the sensor surface.

Further, in one aspect of the present invention, the sensor surface preferably has a rectangular shape in a plan view, and the guide section preferably has a first guide section, a second guide section, a third guide section, and a fourth guide section disposed at four corner sections of the sensor surface in the plan view.

Because of this, the first to fourth guide sections can guide the movement of the ultrasonic probe in the first direction so that the user can precisely move the ultrasonic probe in the first direction.

Further, in one aspect of the present invention, the guide section is preferably configured and arranged to engage with a part of a sheet member for ultrasonic measurement fixed on the target sample to guide a movement in the first direction of the sensor surface of the ultrasonic probe.

Because of this, when the ultrasonic measurement is performed in the case of fixing the sheet member for ultrasonic measurement to the target sample, the guide section can guide the movement of the ultrasonic probe in the first direction of the sensor surface so that the user can precisely move the ultrasonic probe in the longitudinal direction of the sheet member for ultrasonic measurement, for example. Further, the sheet member for ultrasonic measurement can be fixed corresponding to shapes or body types of various parts to be measured so that it can be precisely moved corresponding to a shape of various parts to be measured or a body type.

Further, in one aspect of the present invention, a height of the guide section from the sensor surface is preferably less than or equal to a thickness of the sheet member for ultrasonic measurement.

Because of this, the ultrasonic measurement can be precisely performed because the sensor surface of the ultrasonic probe can contact to the surface of the sheet member for ultrasonic measurement.

Further, in one aspect of the present invention, the resistance force is preferably generated by a friction between the guide section and the target sample.

Because of this, even when the sheet member for ultrasonic measurement is not used, the movement of the ultrasonic probe in the first direction with respect to the target sample can be easier than the movement in the second direction so that the user can precisely move the ultrasonic probe in the first direction.

Further, in one aspect of the present invention, the sensor surface preferably has an elongated shape, and includes a groove portion extending in a longitudinal direction of the sensor surface.

Because of this, a gel applied to the surface of the target sample or the sheet member for ultrasonic measurement can be efficiently collected to an emission surface of the ultrasonic sensor section through the groove portion so that entering air between the ultrasonic sensor section and the target sample or the sheet member for ultrasonic measurement can be prevented.

Further, in one aspect of the present invention, the ultrasonic sensor section is preferably disposed on the sensor surface so that a scanning direction of the ultrasonic transducer device is oriented parallel to a longitudinal direction of the sensor surface, and the guide section is preferably configured and arranged to guide a movement of the ultrasonic probe in the longitudinal direction of the sensor surface.

Because of this, the guide section can guide the movement of the ultrasonic probe in the scanning direction.

An ultrasonic measurement device according to another aspect includes the ultrasonic probe as described above, a transmitting section configured to perform a process of transmitting ultrasonic waves, a receiving section configured to perform a process of receiving ultrasonic echoes, and a processing section configured to perform a process of controlling ultrasonic measurement. The processing section is configured to generate an ultrasonic panoramic image based on a reception signal from the receiving section.

According to another aspect of the present invention, it is possible to easily obtain an ultrasonic panoramic image while precisely moving the ultrasonic probe along a track with a simple configuration.

Another aspect of the present invention relates to an ultrasonic image device including the ultrasonic measurement device described above, and a display section configured to display image data.

### BRIEF DESCRIPTION OF THE DRAWINGS

Referring now to the attached drawings which form a part of this original disclosure:

FIG. 1 is a basic configuration example of an ultrasonic probe.

FIG. 2 is a comparative example of an ultrasonic probe.

FIG. 3A and FIG. 3B are the first configuration example of the ultrasonic probe.

FIG. 4A and FIG. 4B are a modification example of the first configuration example of the ultrasonic probe.

FIG. 5A and FIG. 5B are the second configuration example of an ultrasonic probe.

FIG. 6A and FIG. 6B are the first example of the ultrasonic measurement performed by the ultrasonic probe.

FIG. 7A and FIG. 7B are the second example of the ultrasonic measurement performed by the ultrasonic probe.

FIG. 8 is the third example of the ultrasonic measurement performed by the ultrasonic probe.

FIG. 9 is a diagram describing a movement of the ultrasonic probe.

FIG. 10A and FIG. 10B are a basic configuration of an ultrasonic transducer element.

FIG. 11 is a configuration example of the ultrasonic transducer device.

FIG. 12 is a basic configuration example of an ultrasonic measurement device and an ultrasonic image device.

FIG. 13A and FIG. 13B are a concrete example of the ultrasonic image device.

### DETAILED DESCRIPTION OF EXEMPLARY EMBODIMENTS

Hereinafter, preferred embodiments of the present invention will be described. The present embodiments described below shall not be construed as unreasonably limiting the subject matter of the present invention described in the scope of claims, and all the elements described in the present embodiment are not necessarily essential to the solving means of the present invention.

### 1. Ultrasonic Probe

FIG. 1 is a basic configuration example of an ultrasonic probe 300 of the present embodiment. The ultrasonic probe 300 of the present invention is provided with an ultrasonic sensor section 310, guide section 330 (330-1, 330-2). By the way, the ultrasonic probe 300 of the present embodiment is not limited to the configuration of FIG. 1 so that it is possible to provide various modified embodiments such that a part of the constituent elements may be omitted, it may be replaced to another constituent element, another constituent element may be added, etc.

As shown in FIG. 1, a surface toward the target sample side at the time of measurement by the ultrasonic probe 300 is defined as a sensor surface 320, and a longitudinal direction of the sensor surface 320 is defined as X direction (, first direction), and a direction intersecting the X direction is defined as Y direction (, second direction), and a direction intersecting the X direction and the Y direction and toward the target sample at the time of measurement is defined as Z direction.

The sensor surface 320 is one of the surfaces forming the external surface of the case of the ultrasonic probe 300, and is directed toward the target sample side at the time of ultrasonic measurement. The sensor surface 320 may be a flat surface or a curved surface. For example, the sensor surface 320 is an elongated shape or a rectangular shape in a plan view viewed from the Z direction. The definition of the longitudinal direction of the sensor surface 320 is that for example, the sensor surface 320 is directed along the longitudinal direction when the sensor surface 320 is an elongated shape in the plan view, and the sensor surface 320 is directed along the long side when the sensor surface 320 is a rectangular shape in the plan view. The sensor surface 320 may be, for example, an elliptical shape or a similar shape thereto in the plan view, or a rectangle, which is a shape cutting out the four corners, or a similar shape thereto in the plan view.

The ultrasonic sensor section 310 is provided with an ultrasonic transducer device (not shown) which sends an ultrasonic wave to the target sample (target object) and receives an ultrasonic echo. The ultrasonic sensor section 310 is provided on the sensor surface 320 and is formed along the direction (Y direction) that the scanning direction of the ultrasonic transducer device intersects the longitudinal direction of the sensor surface 320. Alternatively, the ultrasonic sensor section 310 is provided on the sensor surface 320 and is formed along a longitudinal direction (X direction) of the sensor surface 320 that is the scanning direction of the ultrasonic transducer device. For example, as shown in FIG. 1, the ultrasonic sensor section 310 is arranged between the first guide section 330-1 and the second guide section 330-2. The ultrasonic transducer device will be described in detail later.

The guide section 330 is provided on the sensor surface 320, and it is a member projecting to the target sample side (Z direction side) from the sensor surface 320. In the condition that the ultrasonic sensor section 310 is positioned toward the target sample, the guide section 330 provides that a resistance force with respect to the target sample when the sensor surface 320 is contacted to the surface of the target sample and is moved in the X direction is smaller than a resistance force with respect to the target sample in a case of moving in the Y direction. As a result, the movement of the ultrasonic probe 300 in the X direction becomes easier than the movement in the Y direction. The guide section 330 guides the movement of the longitudinal direction of the sensor surface 320. The guide section 330 has a configuration that the longitudinal direction of the sensor surface 320 is defined as the X direction.

For example, as shown in FIG. 1, the guide section 330 has the first guide section 330-1 and the second guide section 330-2 which are provided so as to define the longitudinal direction as the X direction. The first guide section 330-1 is provided in the −Y direction side with respect to the central axis, which passes through the center of the sensor surface 320 and is parallel to the X direction. The second guide section 330-2 is provided in the +Y direction side with respect to the central axis, which passes through the center of the sensor surface 320 and is parallel to the X direction.

As described later, when the ultrasonic measurement is performed with a sheet member for ultrasonic measurement, the guide section 330 is engaged to a part of the sheet member for ultrasonic measurement fixed on the target sample so as to guide the movement of the ultrasonic probe 300 in the longitudinal direction (X direction) of the sensor surface 320. Alternatively, when the ultrasonic measurement is performed without using the sheet member for ultrasonic measurement, by the friction with the target sample, the guide section 330 provides a condition that the movement of the ultrasonic probe 300 in the X direction with respect to the target sample becomes easier than the movement in the Y direction.

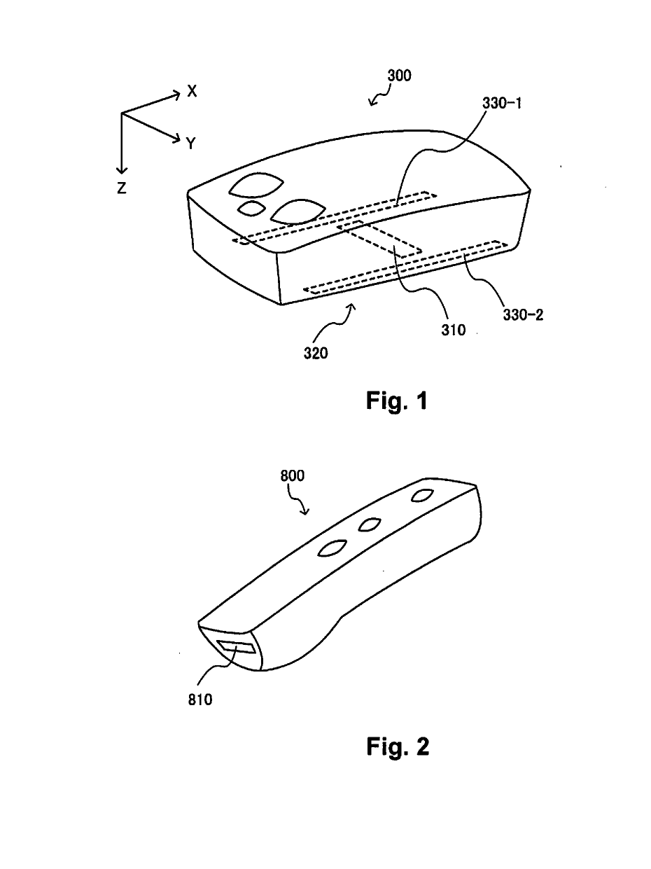

FIG. 2 shows an ultrasonic probe 800 as a comparative example. The ultrasonic probe 800 of the comparative example as shown in FIG. 2 is different from the ultrasonic probe 300 of the present invention so that an ultrasonic sensor section 810 is provided in the end portion of the longitudinal direction of the ultrasonic probe 800. In this type of the ultrasonic probe, it is difficult to stably and vertically hold the probe with respect to the target surface.

On the other hand, as shown in FIG. 1, a shape of the ultrasonic probe 300 of the present embodiment is a mouse-shape so that the ultrasonic sensor section can be provided in a position of the center of gravity of the probe or close to the position of the center of gravity of the probe. In this configuration, it facilitates the measurement by stably holding the probe to the target surface.

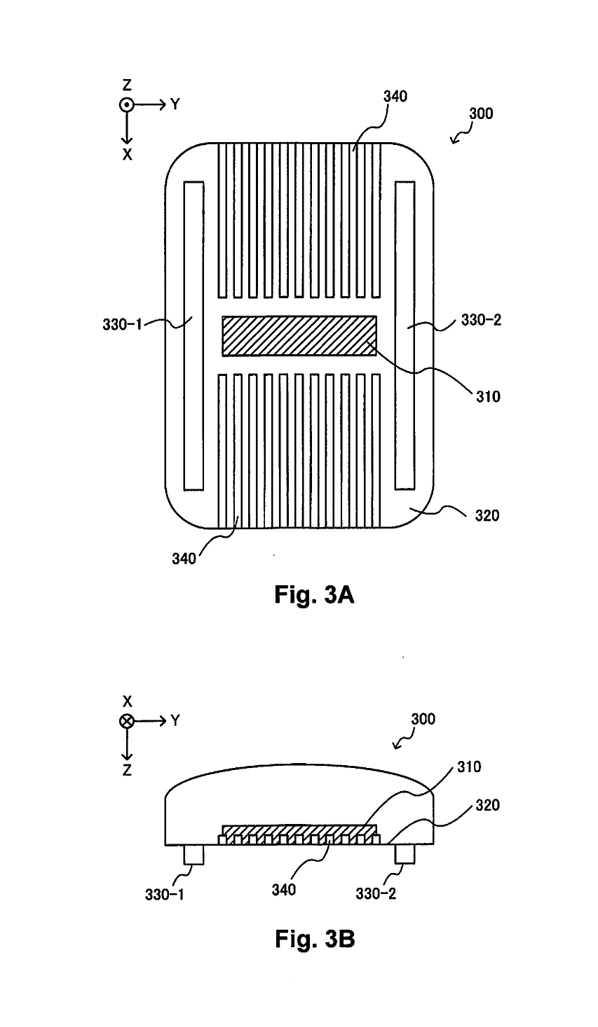

FIG. 3A and FIG. 3B shows the first configuration example of the ultrasonic probe 300 of the present embodiment. The ultrasonic probe 300 of the first configuration example is provided with the ultrasonic sensor section 310, the first and second guide sections 330-1, 330-2, and the groove portion 340. By the way, the ultrasonic probe 300 of the present embodiment is not limited to the configuration of FIG. 3A and FIG. 3B, so that it is possible to provide various modified embodiments such that a part of the constituent elements may be omitted, it may be replaced to another constituent element, another constituent element may be added, etc. For example, the groove portion 340 may be omitted.

FIG. 3A is a diagram viewed from the Z direction side, that is, the sensor surface 320 side that is the surface toward the target sample side at the time of measurement, and FIG. 3B is a schematic diagram viewed from the −X direction side. By the way, the X, Y, Z directions correspond to the definition shown in FIG. 1.

The sensor surface 320 of the ultrasonic probe 300 shown in FIG. 3A has a shape similar to a rectangular shape in a plan view that is viewed from the Z direction side. Specifically, it has a shape that the four corners of the rectangular shape are modified to a rounded shape.

The ultrasonic sensor section 310 has already been described in FIG. 1 so that the detailed description will be omitted here. By the way, the ultrasonic emission surface of the ultrasonic sensor section 310 is not necessary to be provided in the same surface as the sensor surface 320, and a part of the ultrasonic sensor section 310 (e.g., acoustic lens, etc.) may be extended in the Z direction from the sensor surface 320. Alternatively, on the other hand, the ultrasonic emission surface of the ultrasonic sensor section 310 may be reduced in the Z direction from the sensor surface 320.

The first and second guide sections 330-1, 330-2 are provided so as to form the longitudinal direction along the X direction, and guide the movement in the longitudinal direction of the sensor surface 320 of the ultrasonic probe 300. The first guide section 330-1 is provided in the −Y direction side with respect to the central axis which is parallel to the X direction on the sensor surface 320, and the second guide section 330-2 is provided in the +Y direction side with respect to the central axis which is parallel to the X direction on the sensor surface 320.

For example, as shown in FIG. 3A and FIG. 3B, the first and second guide sections 330-1, 330-2 have a length in the X direction and have a width in the Y direction. It is a member projecting in the Z direction (target sample side) and it has a shape similar to a rectangular in a cross-section along the Y direction. By the way, the first and second guide sections 330-1, 330-2 do not have to be the same shape. For example, the length of the first guide section 330-1 may be longer than the length of the second guide section 330-2, and the width of the first guide section 330-1 may be wider than the width of the second guide section 330-2. Alternatively, the cross-sectional shape of the first guide section 330-1 and the second guide section 330-2 may be different. For example, the thickness of the first guide section 330-1 in the X direction may be thicker than the thickness of the second guide section 330-2 in the Z direction.

The groove portion 340 has a length in the X direction, a width in the Y direction, and a depth in the −Z direction. The groove portion is an opening on the sensor surface 320. On the sensor surface 320, it is provided that the longitudinal direction is formed in the X direction (, the first direction), that is, the longitudinal direction of the groove portion 340 is provided so as to be formed in the longitudinal direction of the sensor surface 320. The groove portion 340 has a concave shape in the cross-section along the Y direction. For example, as shown in FIG. 3A and FIG. 3B, a plurality of groove portions 340 may be respectively provided in a region of +X direction side and a region of −X direction side with respect to the ultrasonic sensor section 310 on the sensor surface 320. The number of the groove portion 340 is not limited to the number in the drawings. Each of the plurality of groove portions 340 may be the same shape, or for example, the length, width, depth, etc. of each groove portion may be different, respectively. Further, the respective groove portions may not have to be parallel each other.

By providing the groove portion 340, when the ultrasonic measurement is performed while moving the ultrasonic probe 300 in the X direction, a gel applied to the target surface or the sheet member for ultrasonic measurement can be efficiently collected to the emission surface of the ultrasonic sensor section 310 through the groove portion 340. Because of this, entering air between the ultrasonic sensor section 310 and the target sample or the sheet member for ultrasonic measurement can be prevented.

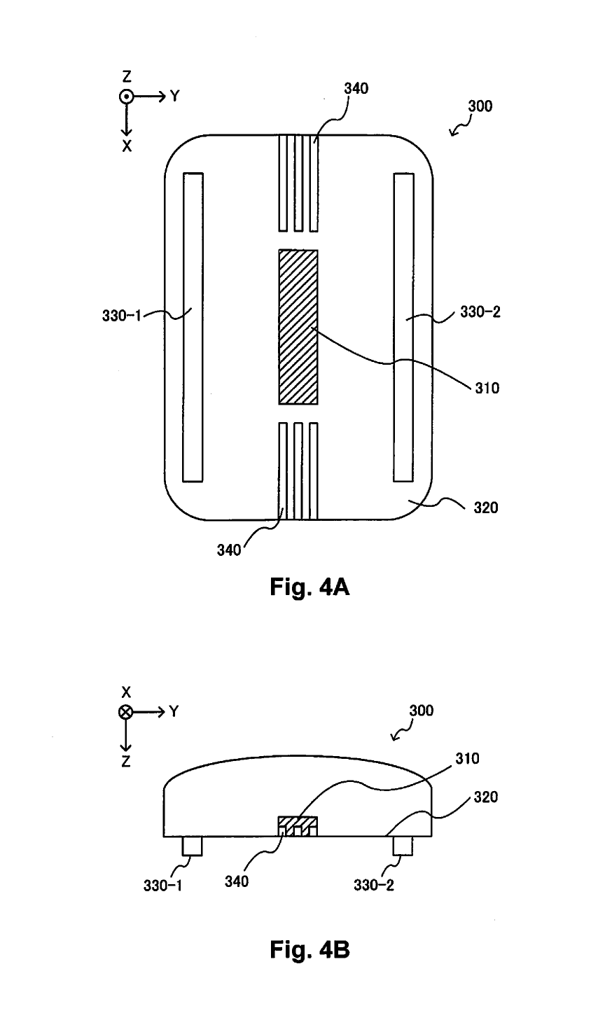

FIG. 4A and FIG. 4B shows a modification example of the first configuration example of the ultrasonic probe 300 of the present embodiment. In the modification example of the first configuration example, the ultrasonic sensor section 310 is provided on the sensor surface 320 so that the scanning direction of the ultrasonic transducer device is parallel to the longitudinal direction of the sensor surface 320. The first and second guide sections 330-1, 330-2 guide the movement in the longitudinal direction of the sensor surface 320 of the ultrasonic probe 300. That is, it can guide the movement of the ultrasonic probe 300 in the scanning direction.

According to the modification example of the first configuration example of the ultrasonic probe 300 of the present embodiment, the ultrasonic measurement is performed while moving the ultrasonic probe 300 in the scanning direction along a desired track so that a plurality of ultrasonic images along the desired track can be easily obtained. As a result, for example, it is possible to obtain an ultrasonic panoramic image along the desired track.

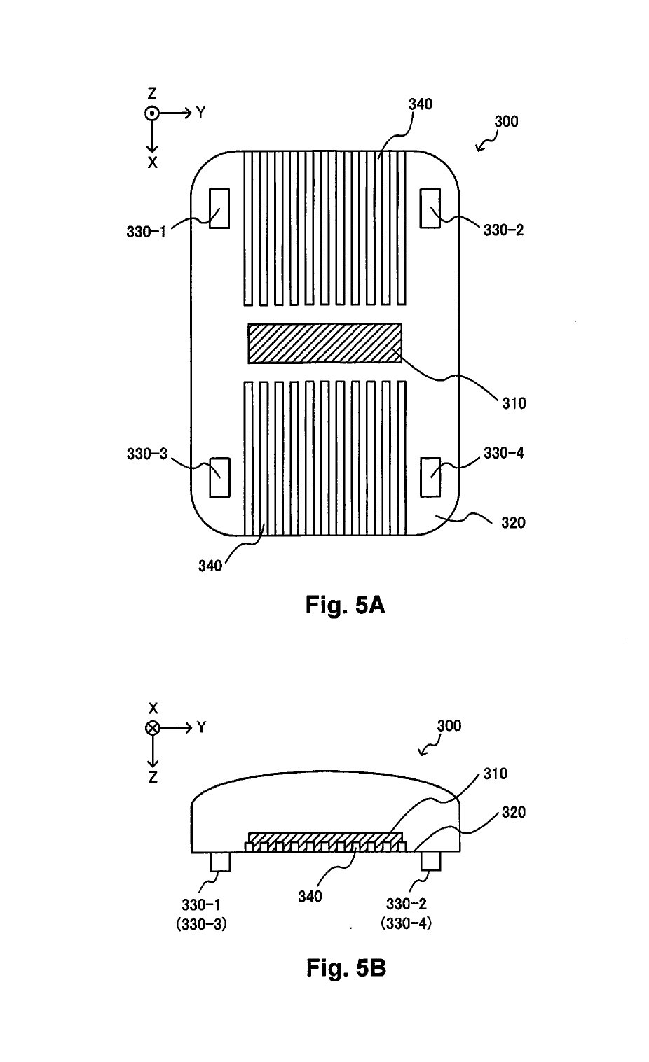

FIG. 5A and FIG. 5B shows the second configuration example of the ultrasonic probe 300 of the present embodiment. The ultrasonic probe 300 of the second configuration example is provided with the ultrasonic sensor section 310, the first to fourth guide sections 330-1 to 330-4, and the groove portion 340. By the way, the ultrasonic probe 300 of the present embodiment is not limited to the configuration of FIG. 5A and FIG. 5B so that it is possible to provide various modified embodiments such that a part of the constituent elements may be omitted, it may be replaced to another constituent element, another constituent element may be added, etc. For example, the groove portion 340 may be omitted.

FIG. 5A is a diagram viewed from the Z direction side, that is, the sensor surface 320 side that is the surface toward the target sample side at the time of measurement. FIG. 5B is a schematic diagram viewed from the −X direction side. By the way, the respective directions of X, Y, Z correspond to the definition shown in FIG. 1.

The ultrasonic sensor section 310 has already been described in FIG. 1, FIG. 3A, and FIG. 3B so that the detailed description will be omitted here.

The first to fourth guide sections 330-1 to 330-4 are provided in the first to fourth corner parts of the sensor surface 320, and guide the movement in the longitudinal direction (X direction) of the sensor surface 320 of the ultrasonic probe 300.

In the sensor surface 320, the range of X in the X-condition in the region where the ultrasonic sensor section 310 is provided is defined as xa≦x≦xb and when the range of Y in the Y-condition is defined as ya≦y≦yb the first corner part is the region that is x<xa and y<ya in the sensor surface 320. Also, the second corner part is the region that is x<xa and y>yb in the sensor surface 320. The third corner part is the region that is x>xb and y<ya in the sensor surface 320. The fourth corner part is the region that is x>xb and y>yb in the sensor surface 320.

For example, as shown in FIG. 5A and FIG. 5B, the first to fourth guide sections 330-1 to 330-4 have a length in the X direction and a width in the Y direction, and it is a member projecting to the Z direction (target sample side) from the sensor surface 320 so that the cross-section along the Y direction is a rectangular shape or similar to the shape. By the way, the shape of the first to fourth guide sections 330-1 to 330-4 is not limited to the shape in the drawing so that it may be, for example, a cylindrical shape or an elliptic shape. Also, the first to fourth guide sections 330-1 to 33-4 may be different shape, respectively. Further, the first to fourth guide sections 330-1 to 330-4 may not have to be symmetrically arranged each other with respect to the central axis, which passes through the center of the sensor surface 320 and is parallel to the X direction, or the central axis, which passes through the center of the sensor surface 320 and is parallel to the Y direction.

The groove portion 340 has already been described in FIG. 3A and FIG. 3B so that the detailed description will be omitted here.

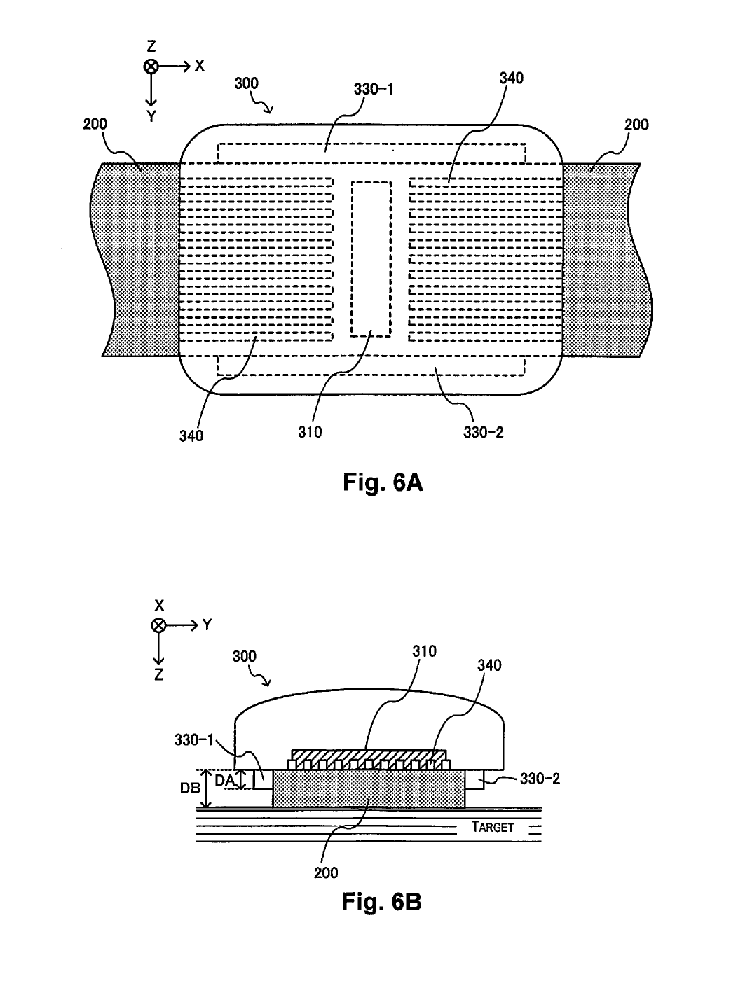

FIG. 6A and FIG. 6B shows the first example of the ultrasonic measurement performed by the ultrasonic probe 300 of the present embodiment. Here, it is shown in the case that the ultrasonic probe 300 of the first configuration example and the sheet member for ultrasonic measurement 200 are used. FIG. 6A is a diagram viewed from −Z direction side, that is, it is viewed from the opposite side of the sensor surface 320. FIG. 6B is a schematic diagram viewed from −X direction side.

The sheet member for ultrasonic measurement 200 is configured by an ultrasonic transmissive medium that transmits an ultrasonic wave, and it is fixed on the surface of the target sample at the time of the ultrasonic measurement. The ultrasonic probe 300 emits an ultrasonic wave to the target sample (target object) through the sheet member for ultrasonic measurement 200.

The sheet member for ultrasonic measurement 200 is a sheet which transmits an ultrasonic wave, and is provided between the ultrasonic probe 300 and the target sample to secure an acoustic matching (acoustic impedance matching) between the ultrasonic sensor section 310 and the target sample at the time of ultrasonic measurement.

The first and second guide sections 330-1, 330-2 are engaged with a part of the sheet member for ultrasonic measurement fixed on the target sample, and guide the movement in the longitudinal direction (X direction) of the sensor surface 320 of the ultrasonic probe 300. Specifically, the first guide section 330-1 is engaged with the end portion of −Y direction side of the sheet member for ultrasonic measurement 200, and the second guide section 330-2 is engaged with the end portion of +Y direction side of the sheet member for ultrasonic measurement 200 so that it is possible to guide the movement in the longitudinal direction (X direction) of the sensor surface 320 of the ultrasonic probe 300. That is, the surface (edge face) of the end portion of −Y direction side of the sheet member for ultrasonic measurement 200 and the surface of the first guide section 330-1, which is opposed to the edge face, are contacted, and the surface of the end portion of +Y direction side of the sheet member for ultrasonic measurement 200 and the surface of the second guide section 330-2, which is opposed to the surface of the end portion, are contacted so that the movement of the ultrasonic probe 300 in the Y direction, which is a direction perpendicular to the surface, can be limited.

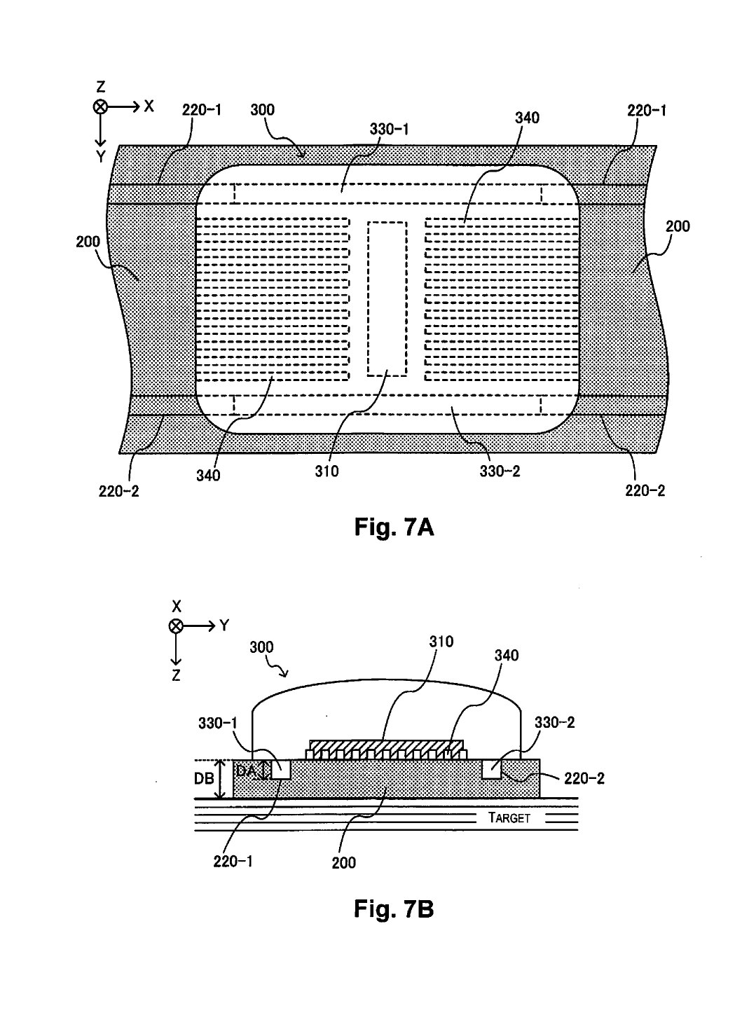

A part of the sheet member for ultrasonic measurement 200 is a portion of the sheet member for ultrasonic measurement 20, and it is a portion that guides the movement in the longitudinal direction (X direction) of the sensor surface 320 of the ultrasonic probe 300 by engaging with the guide section 330, or a portion that limits the movement of the Y direction of the ultrasonic probe 300. For example, it is a portion contacting and fitting with the projecting portion of the guide section 330, a portion guiding the movement in the longitudinal direction (X direction) of the sensor surface 320 of the ultrasonic probe 300 by fitting, or a portion limiting the movement in the Y direction of the ultrasonic probe 300. Specifically, it may be the end portion shown in FIG. 6A and FIG. 6B, or it may be the groove portion shown in FIG. 7A and FIG. 7B.

The height DA from the sensor surface 320 of the first and second guide sections 330-1, 330-2 is less than or equal to the thickness DB of the sheet member for ultrasonic measurement 200. By the way, the height DA of the first and second guide sections 330-1, 330-2 may be different.

FIG. 6A and FIG. 6B shows when the first configuration example (FIG. 3A, FIG. 3B) of the ultrasonic probe 300 is used as an example, but it is also the same manner when the modified example of the first configuration example (FIG. 4A, FIG. 4B) is used. Also, it is the same manner when the second configuration example (FIG. 5A, FIG. 5B) is used. That is, the first and third guide sections 330-1, 33-3 are engaged with the end portion of the −Y direction side of the sheet member for ultrasonic measurement 200, and the second and fourth guide sections 330-2, 330-4 are engaged with the end portion of the +Y direction side of the sheet member for ultrasonic measurement 200 so that it is possible to guide the movement in the longitudinal direction (X direction) of the sensor surface 320 of the ultrasonic probe 300.

The end portion of the sheet member for ultrasonic measurement 200 is a portion including the surface of +Y direction side and its vicinity or a portion including the surface of −Y direction side and its vicinity among the six surfaces that forms the outer surface of the sheet member for ultrasonic measurement 200.

FIG. 7A and FIG. 7B shows the second example of the ultrasonic measurement performed by the ultrasonic probe 300 of the present embodiment. Here, it is shown in the case that the ultrasonic probe 300 of the first configuration example and the sheet member for ultrasonic measurement 200 are used. FIG. 7A is a diagram viewed from −Z direction side, that is, it is viewed from the opposite side of the sensor surface 320. FIG. 7B is a schematic diagram viewed from −X direction side.

The sheet member for ultrasonic measurement 200 shown in FIG. 7A and FIG. 7B includes the first and second groove portions 220-1, 220-2 provided along the longitudinal direction (X direction) of the sheet member for ultrasonic measurement 200. The first and second groove portions 220-1, 220-2 are the groove portion guiding the movement of the ultrasonic probe 300.

The first and second groove portions 220-1, 220-2 are provided in the ultrasonic probe 200 side of the surface of the sheet member for ultrasonic measurement 200, and have a length in the X direction, a width in the Y direction, and a depth in the Z direction, and is the groove portion that opens in the ultrasonic probe 300 side of the surface of the sheet member for ultrasonic measurement 200. The first and second groove portions 220-1, 220-2 have a concave shape in the cross-section along the Y direction. The first groove portion 220-1 is provided in the −Y direction side with respect to the central axis along the longitudinal direction (X direction) of the sheet member for ultrasonic measurement 200, and the second groove portion 220-2 is provided in the +Y direction side with respect to the central axis along the longitudinal direction (X direction) of the sheet member for ultrasonic measurement 200.

The first and second guide sections 330-1, 330-2 are engaged with a part of the sheet member for ultrasonic measurement fixed on the target sample, and guide the movement in the longitudinal direction (X direction) of the sensor surface 320 of the ultrasonic probe 300. Specifically, the first guide section 330-1 is fitted (engaged) with the first groove portion 220-1 of the sheet member for ultrasonic measurement 200 and the second guide section 330-2 is fitted with the second groove portion 220-2 of the sheet member for ultrasonic measurement 200 so that it is possible to guide the movement in the longitudinal direction (X direction) of the sensor surface 320 of the ultrasonic probe 300. Alternatively, it is possible to limit the movement of the ultrasonic probe 300 in the Y direction with respect to the target sample. That is, the surface of the first guide section 330-1 and the surface of the first groove portion 220-1, which is opposed to the surface of the first guide section, are contacted, and the surface of the second guide section 330-2 and the surface of the second groove portion 220-2, which is opposed to the surface of the second guide section, are contacted so that it is possible to guide the movement in the longitudinal direction (X direction) of the sensor surface 320 of the ultrasonic probe 300.

The height DA from the sensor surface 320 of the first and second guide sections 330-1, 330-2 is less than or equal to the thickness DB of the sheet member for ultrasonic measurement 200. By the way, the height DA of the first and second guide sections 330-1, 330-2 may be different, respectively. The height DA of the first and second guide sections 330-1, 330-2 is the length from the sensor surface 320 to the projected edge in the Z direction.

FIG. 7A and FIG. 7B shows the case of the first configuration example (FIG. 3A and FIG. 3B) of the ultrasonic probe 300 as an example, but the case of the modified example (FIG. 4A and FIG. 4B) of the first configuration example is in the same manner. Also, the case of the second configuration example (FIG. 5A and FIG. 5B) is in the same manner. That is, the first and third guide sections 330-1, 330-3 are engaged with the first groove portion 220-1 of the sheet member for ultrasonic measurement 200, and the second and fourth guide sections 330-2, 330-4 are engaged with the second groove portion 220-2 of the sheet member for ultrasonic measurement 200 so that it is possible to guide the movement in the longitudinal direction (X direction) of the sensor surface 320 of the ultrasonic probe 300.

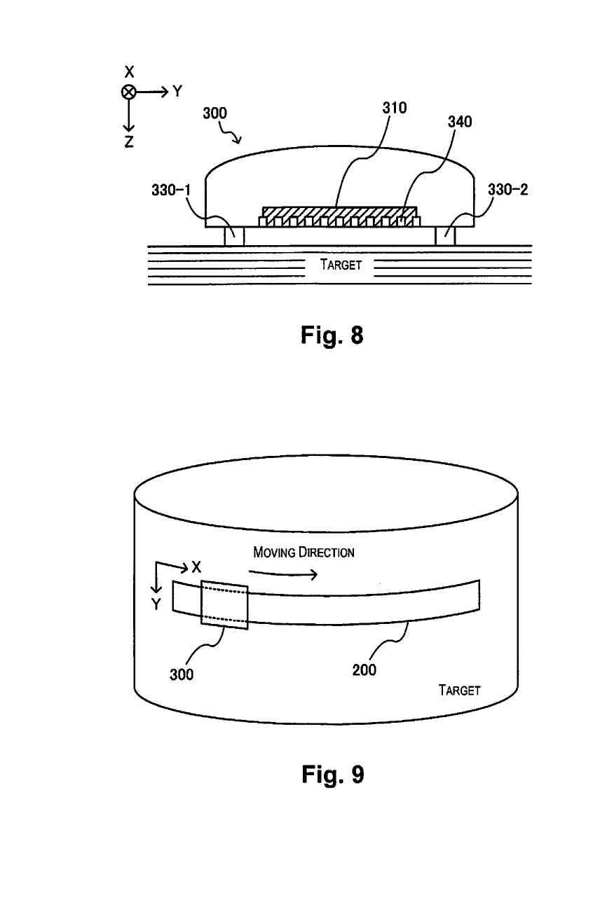

FIG. 8 shows the third example of the ultrasonic measurement performed by the ultrasonic probe 300 of the present invention. Here, it is shown in the case that the sheet member for ultrasonic measurement 200 is not used. FIG. 8 is a schematic diagram viewed from the −X direction side.

By the friction with the target sample, the first and second guide sections 330-1, 330-2 provide a condition that the resistance force with respect to the target sample when the ultrasonic sensor section 310 is moved in the X direction by contacting the sensor surface 320 to the surface of the target sample becomes smaller than the resistance force with respect to the target sample in the case of moving in the Y direction. As a result, by the friction with the target sample, the first and second guide sections 330-1, 330-2 provide a condition that the movement of the ultrasonic probe 300 in the X direction with respect to the target sample can be easier than the movement in the Y direction. In the case of the second configuration example of the ultrasonic probe 300, in the same manner, the first to fourth guide sections 330-1 to 330-4 provide a condition that the movement of the ultrasonic probe 300 in the X direction with respect to the target sample can be easier than the movement in the Y direction by the friction with the target sample.

By the way, when the sheet member for ultrasonic measurement 200 is not used, by applying the gel on the surface of the target sample, entering air between the target sample and the ultrasonic sensor section 310 can be prevented. As described above, by providing the groove portion 340 on the sensor surface 320 of the ultrasonic probe 300, the gel applied on the surface of the target sample can be efficiently collected to the emission surface of the ultrasonic sensor section 310 through the groove portion 340.

FIG. 9 is an explanatory diagram showing the movement of the ultrasonic probe 300 guided by the sheet member for ultrasonic measurement 200. The longitudinal direction of the sheet member for ultrasonic measurement 200 is defined as the X direction.

As described above, the guide section 330 provided in the ultrasonic probe 300 is engaged with a part of the sheet member for ultrasonic measurement 200 fixed on the target sample so as to guide the movement in the longitudinal direction (X direction) of the sensor surface 320 of the ultrasonic probe 300.

As shown in FIG. 9, the user fixes the sheet member for ultrasonic measurement 200 on a measurement target region (region of interest) of the target sample and the ultrasonic probe 300 is set on the region. At this time, the scanning direction or the slice direction is set so as to be parallel to the X direction. The movement of the ultrasonic probe 300 in the Y direction is limited, but the movement in the X direction is not limited. That is, the ultrasonic probe 300 can be freely moved in the longitudinal direction of the sheet member for ultrasonic measurement 200. As a result, the ultrasonic probe 300 can be securely moved along the track defined by the sheet member for ultrasonic measurement 200. Also, the sheet member for ultrasonic measurement 200 can be fixed corresponding to a shape of the target sample, and the like, so that the ultrasonic probe 300 can be precisely moved corresponding to a shape of various parts to be measured or a body type of the target sample.

Because of this, the ultrasonic measurement can be performed while moving the ultrasonic probe 300 along the predetermined track so that it is possible to easily obtain a plurality of ultrasonic images along the predetermined track on, for example, the measurement target region. Further, it is possible to obtain an ultrasonic panoramic image or a three-dimensional ultrasonic image based on the plurality of ultrasonic images along the predetermined track.

As described above, according to the ultrasonic probe 300 of the present embodiment, by the guide section 330 provided on the sensor surface 320, it can guide the movement in the longitudinal direction of the sensor surface 320 of the ultrasonic probe 300. In addition, by using the sheet member for ultrasonic measurement, it can guide the movement of the ultrasonic probe in the longitudinal direction of the sheet member for ultrasonic measurement. As a result, obtaining a plurality of ultrasonic images while the user moves the ultrasonic probe in the longitudinal direction of the sheet member for ultrasonic measurement, that is, precisely moving along the predetermined track can be easily performed with a simple configuration. Moreover, it is possible to obtain an ultrasonic panoramic image or a three-dimensional ultrasonic image based on the plurality of ultrasonic images obtained such way.

### 2. Ultrasonic Transducer Device

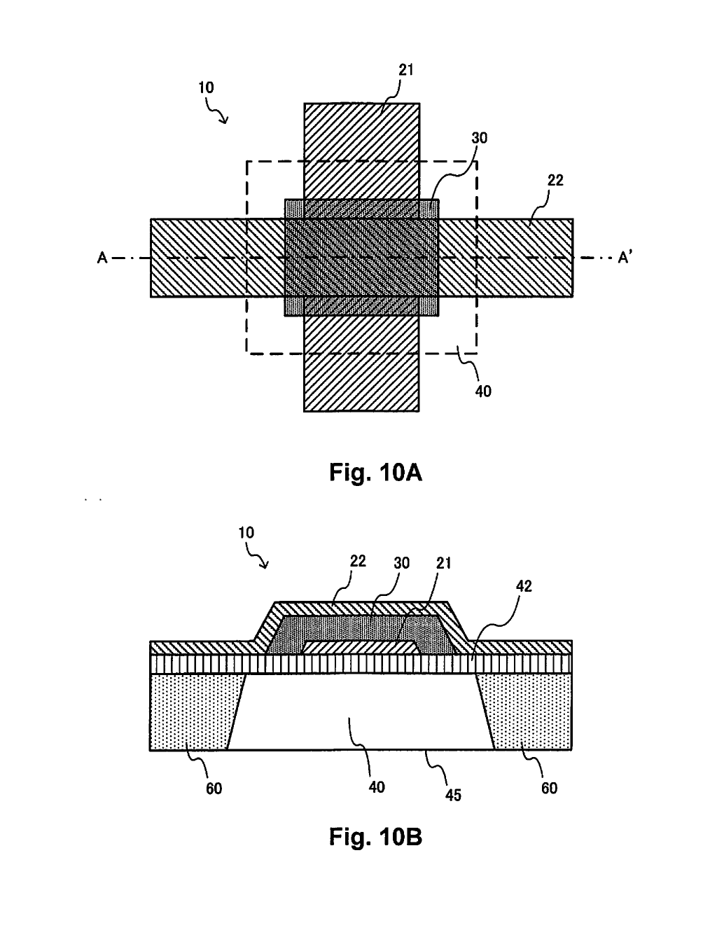

The ultrasonic sensor section 310 of the ultrasonic probe 300 of the present embodiment has an ultrasonic transducer device 312. FIG. 10A and FIG. 10B show a basic configuration example of an ultrasonic transducer element 10(thin film piezoelectric ultrasonic transducer element). The ultrasonic transducer element 10 of the present embodiment is provided with a vibrating membrane 42 and a piezoelectric element part. The piezoelectric element part is provided with a first electrode layer 21, a piezoelectric element 30, and a second electrode layer 22. By the way, the ultrasonic transducer element 10 of the present embodiment is not limited to the configuration of FIG. 10A and FIG. 10B so that it is possible to provide various modified embodiments such that a part of the constituent elements may be omitted, it may be replaced to another constituent element, another constituent element may be added, etc.

FIG. 10A is a diagram showing a plane view of the ultrasonic transducer element 10 formed on a substrate 60 (silicon substrate) which is viewed from a direction perpendicular to the substrate in the element formation surface side. FIG. 10B is a diagram showing a cross-sectional view along A-A′ of FIG. 10A.

The first electrode layer 21 (lower electrode) is formed by, for example, a metallic thin film on the upper layer of the vibrating membrane 42. The first electrode layer 21 is extended outward of the element formation region as shown in FIG. 10A, and it may be a wire connected to the adjacent ultrasonic transducer element 10.

The piezoelectric layer 30 is formed by, for example, a lead zirconate titanate (PZT) thin film, and it is provided to cover at least a part of the first electrode layer 21. By the way, the material of the piezoelectric layer 30 is not limited to PZT so that for example, lead titanate (PbTiO₃), lead zirconate (PbZrO₃), lead lanthanum titanate ((Pb, La)TiO₃) may be used.

The second electrode layer 22 (upper electrode) is formed by, for example, a metal thin film, and it is provided to cover at least a part of the piezoelectric layer 30. The second electrode layer 22 is extended to the outside of the element formation area as shown in FIG. 10A, and it may be a wire connected to the adjacent ultrasonic transducer element 10.

The vibrating membrane 42 (membrane) is provided to cover the opening 45 by two layers structure, for example, SiO₂ thin film and ZrO₂ thin film. The vibrating membrane 42 supports the piezoelectric layer 30, and the first and second electrode layers 21, 22, and it vibrates in accordance with the expansion and contraction of the piezoelectric layer 30 so as to generate the ultrasonic wave.

The opening 45 is arranged in the substrate 60. A cavity region 40 of the opening 45 is formed by etching such as Reactive Ion Etching (RIE), etc. from the back surface (the surface where an element is not formed) side of the substrate 60. A resonance frequency of the ultrasonic wave is determined by the size of the vibrating membrane 42 which enables to be vibrated by forming the cavity region 40, and the ultrasonic wave is radiated to the piezoelectric layer 30 side (forward direction from the back surface on a page in FIG. 10A).

The lower electrode of the ultrasonic transducer element 10 is formed by the first electrode layer 21, and the upper electrode is formed by the second electrode layer 22. Specifically, a portion that is covered by the piezoelectric film 30 within the first electrode layer 21 forms a lower electrode, and a portion that covers the piezoelectric film 30 within the second electrode layer 22 forms an upper electrode. That is, the piezoelectric film 30 is provided between the lower electrode and the upper electrode.

The piezoelectric film 30 stretches in-plane direction by applying a voltage between the lower electrode and the upper electrode, that is, the first electrode layer 21 and the second electrode layer 22. The ultrasonic transducer element 10 uses a monomorph (unimorph) structure in which a thin piezoelectric element part and the vibrating membrane are bonded together, and when the piezoelectric element part stretches in-plane, the dimension of the bonded vibrating membrane 42 is unchanged so as to occur warping. Accordingly, by applying alternating voltage to the piezoelectric film 30, the vibrating membrane 42 vibrates in a film thickness direction so that the ultrasonic wave is radiated by the vibration of the vibrating membrane 42. The voltage applied to the piezoelectric film 30 is, for example, 10V to 30V, and the frequency is, for example, 1 MHz to 10 MHz.

The driving voltage of the ultrasonic transducer element of bulk is approximately 100V from a peak to a peak, and on the other hand, in the thin film piezoelectric ultrasonic transducer element shown in FIG. 10A and FIG. 10B, the driving voltage can be approximately 10 to 30V from a peak to a peak.

The ultrasonic transducer element 10 operates as a reception element receiving an ultrasonic echo where the radiated ultrasonic wave is returned by the reflection at the target object. The vibrating membrane 42 is vibrated by the ultrasonic echo and the pressure is applied to the piezoelectric film 30 by this vibration so that the voltage is generated between the lower electrode and the upper electrode. This voltage can be taken out as a reception signal.

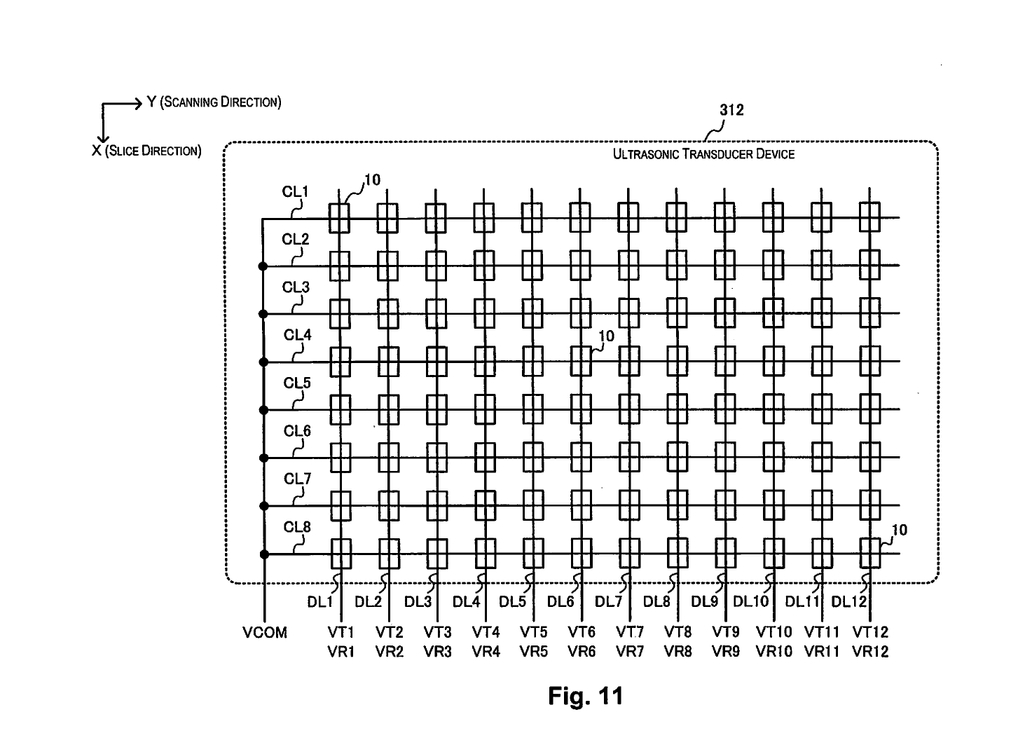

FIG. 11 shows a configuration example of the ultrasonic transducer device 312 provided in the ultrasonic probe 300 of the present embodiment. The ultrasonic transducer device 312 of the present configuration example includes a plurality of ultrasonic transducer elements 10 arranged in an array pattern, the 1st to n-th driving electrode wires DL1 to DLn (n is an integer of more than or equal to 2), and the 1st to m-th (m is an integer of more than or equal to 2) common electrode wires CL1 to CLm. FIG. 11 shows the case of m=8, n=12, but it may be more than the values. By the way, the ultrasonic transducer device 312 of the present embodiment is not limited to the configuration of FIG. 11 so that it is possible to provide various modified embodiments such that a part of the constituent elements may be omitted, it may be replaced another constituent element, another constituent element may be added, etc.

The plurality of ultrasonic transducer elements 10 are arranged in a matrix pattern of m rows and n columns. For example, as shown in FIG. 11, 8 rows in the X direction and 12 columns in the Y direction, which intersects the X direction, are arranged. The ultrasonic transducer element 10 may be the configuration, for example, as shown in FIG. 10A and FIG. 10B.

The 1^{st} to 12^{th} driving electrode wires DL1 to DL12 (, n-th) are arranged in the X direction. The j-th driving electrode wire DLj (j is an integer of 1≦j≦12) among the 1^{st} to 12^{th} driving electrode wires DL1 to DL12 is connected to the first electrode provided in each ultrasonic transducer element 10 arranged in the j-th column.

In a transmission period that an ultrasonic wave is radiated, the 1^{st} to 12^{th} transmission signals VT1 to VT12 outputted from a transmitting section 110, which will be described later, are supplied to each ultrasonic transducer element 10 through the driving electrode wires DL1 to DL12. Also, in a reception period that an ultrasonic echo is received, the reception signals VR1 to VR12 from the ultrasonic transducer element 10 are outputted to a reception section 120, which will be described later, through the driving electrode wires DL1 to DL12.

The 1^{st} to 8^{th} common electrode wires CL1 to CL8 (, m-th) are wired in the Y direction. The second electrode provided in the ultrasonic transducer element 10 is connected to any of the 1^{st} to m-th common electrode wires CL1 to CLm. Specifically, for example, as shown in FIG. 11, the i-th common electrode wire CLi (i is an integer of 1≦i≦8) of the 1^{st} to 8^{th} common electrode wires CL1 to CL8 is connected to the second electrode in each ultrasonic transducer element 10 arranged in the i-th column.

In the 1^{st} to 8^{th} common electrode wires CL1 to CL8, a common voltage VCOM is supplied. This common voltage may be a fixed DC voltage, and it does not have to be OV, that is, ground potential (grounding potential).

For example, for the 1st row and the 1st column of the ultrasonic transducer element 10, the first electrode is connected to the driving electrode wire D1, and the second electrode is connected to the 1^{st} common electrode wire CL1. Also, for example, for the 4^{th} row and the 6th column of the ultrasonic transducer element 10, the first electrode is connected to the 6^{th} driving electrode wire DL6, and the second electrode is connected to the 4^{th} common electrode wire CL4.

By the way, the arrangement of the ultrasonic transducer elements 10 is not limited to the matrix arrangement of m-rows and n-columns as shown in FIG. 11. For example, m ultrasonic transducer elements 10 are arranged in the odd number of columns, and m−1 ultrasonic transducer elements 10 are arranged in the even number of columns. That is, it may be a zigzag pattern.

The element included in the ultrasonic transducer device 312 is not limited to the aforementioned thin film piezoelectric ultrasonic transducer element, and for example, it may be a bulk piezoelectric ultrasonic transducer element or an ultrasonic transducer element (CMUT: Capacitive Micromachined Ultrasonic Transducer).

### 3. Ultrasonic Measurement Device and Ultrasonic Image Device

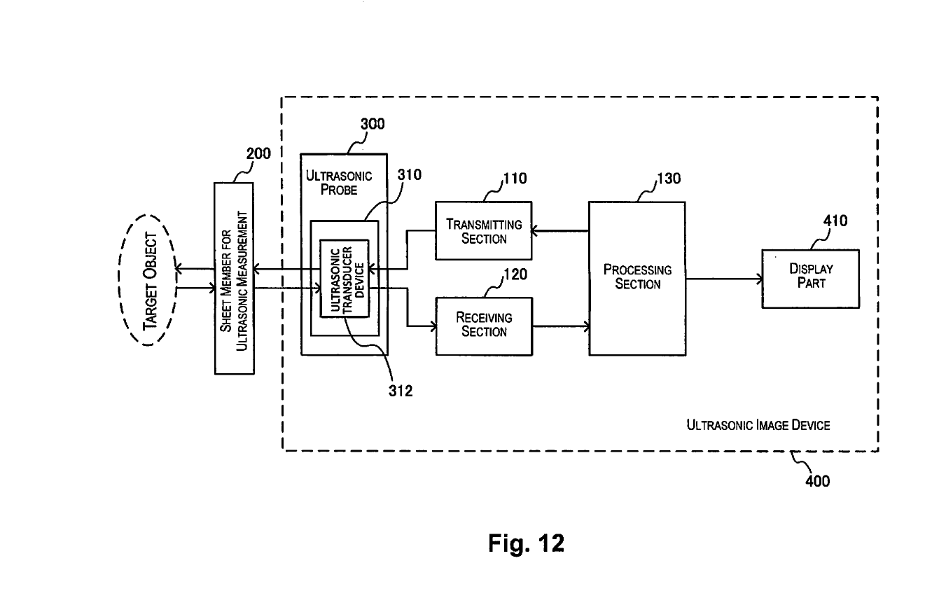

FIG. 12 shows a basic configuration example of an ultrasonic measurement device 100 and the ultrasonic image device 400 of the present embodiment. The ultrasonic measurement device 100 is provided with the ultrasonic probe 300, the transmitting section 110, the reception section 120, and the processing section 130. The ultrasonic image device 400 is provided with the ultrasonic measurement device 100 and the display section 410. By the way, the ultrasonic measurement device 100 and the ultrasonic image device 400 of the present embodiment are not limited to the configuration of FIG. 12 so that it is possible to provide various modified embodiments such that a part of the constituent elements may be omitted, it may be replaced to another constituent element, another constituent element may be added, etc.

The ultrasonic probe 300 has already been described so that the detailed description will be omitted. By the way, the ultrasonic probe 300 may perform the ultrasonic measurement through the sheet member for ultrasonic measurement 200 as shown in FIG. 12, and alternatively, it may perform the ultrasonic measurement without the sheet member for ultrasonic measurement 200.

The transmitting section 110 performs a transmission process of an ultrasonic wave. Specifically, the transmitting section 110 outputs a transmission signal (driving signal) to the ultrasonic probe 300, and the ultrasonic transducer device 312 provided in the ultrasonic probe 300 converts the transmission signal, which is an electronic signal, to the ultrasonic wave so that the ultrasonic wave is emitted to the target object. At least a part of the transmitting section 110 may be provided in the ultrasonic probe 300.

The reception section 120 performs a reception processing of the ultrasonic echo. Specifically, the ultrasonic transducer device 312 provided in the ultrasonic probe 300 converts the ultrasonic echo, which comes from the target object, to the electronic signal. And, the reception section 120 performs the reception processing such as amplification of the reception signal (analog signal), which is the electronic signal from the ultrasonic transducer device 312, detection, A/D conversion, position adjustment, etc., and outputs the reception signal (digital data), which is a signal after the reception processing, to the processing section 130. At least a part of the reception section 120 may be provided in the ultrasonic probe 300.

The processing section 130 performs an image data generation processing based on the control processing of the ultrasonic measurement or the reception signal from the reception section 120. The generated image data is outputted to the display section 410.

The display section 410 is a display device such as, for example, a liquid crystal display, organic electroluminescence display, etc., and displays the image data for display from the processing section 130.

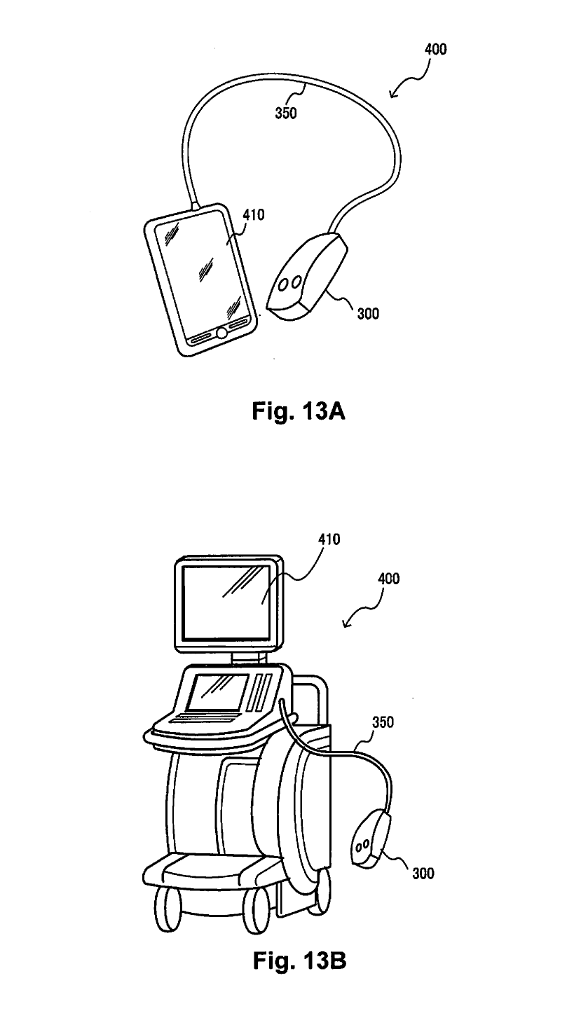

FIG. 13A and FIG. 13B shows a concrete configuration example of the ultrasonic image device 400 of the present embodiment. FIG. 13A shows a portable type of the ultrasonic image device 400, and FIG. 13B shows a stationary type of the ultrasonic image device 400.

The ultrasonic prove 300 is connected to the ultrasonic image device main body by the cable 350. The display section 410 displays the image data for display.

By the way, the present embodiments were described above in detail, but it will be apparent to those skilled in the art that various modifications can be made in a scope not substantially deviating from the subject matter and the effect of the present invention. Therefore, such changes and modifications are included in the scope of the invention. For example, the terms used in the specification or the drawings at least once together with a different term having a broader or similar meaning can be replaced with the different term in any portion of the specification or the drawings. Also, the configuration and the operation of the ultrasonic probe, the ultrasonic measurement device, and the ultrasonic measurement device and the ultrasonic image device are also not limited to the description in the present embodiments so that various modifications can be possible.

### GENERAL INTERPRETATION OF TERMS

In understanding the scope of the present invention, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” or “element” when used in the singular can have the dual meaning of a single part or a plurality of parts. Finally, terms of degree such as “substantially”, “about” and “approximately” as used herein mean a reasonable amount of deviation of the modified term such that the end result is not significantly changed. For example, these terms can be construed as including a deviation of at least ±5% of the modified term if this deviation would not negate the meaning of the word it modifies.

While only selected embodiments have been chosen to illustrate the present invention, it will be apparent to those skilled in the art from this disclosure that various changes and modifications can be made herein without departing from the scope of the invention as defined in the appended claims. Furthermore, the foregoing descriptions of the embodiments according to the present invention are provided for illustration only, and not for the purpose of limiting the invention as defined by the appended claims and their equivalents.

What is claimed is:

1. An ultrasonic probe comprising:
an ultrasonic sensor section having an ultrasonic transducer device; and
a guide section disposed on a sensor surface on which the ultrasonic sensor section is provided, the guide section being configured and arranged such that a resistance force with respect to a target sample when the ultrasonic probe is moved in a first direction while the sensor surface contacts a surface of the target sample as the ultrasonic sensor section faces the target sample is smaller than the resistance force with respect to the target sample when the ultrasonic probe is moved in a second direction intersecting the first direction.

2. The ultrasonic probe according to claim 1, wherein
the sensor surface has an elongated shape in a plan view, and
a longitudinal direction of the sensor surface is defined as the first direction.

3. The ultrasonic probe according to claim 1, wherein
the guide section has a first guide section and a second guide section with a longitudinal direction of the sensor surface is defined as the first direction, and
the ultrasonic sensor section is disposed between the first guide section and the second guide section.

4. The ultrasonic probe according to claim 1, wherein
the sensor surface has a rectangular shape in a plan view, and
the guide section has a first guide section, a second guide section, a third guide section, and a fourth guide section disposed at four corner sections of the sensor surface in the plan view.

5. The ultrasonic probe according to claim 1, wherein
the guide section is configured and arranged to engage with a part of a sheet member for ultrasonic measurement fixed on the target sample to guide a movement in the first direction of the sensor surface of the ultrasonic probe.

6. The ultrasonic probe according to claim 5, wherein
a height of the guide section from the sensor surface is less than or equal to a thickness of the sheet member for ultrasonic measurement.

7. The ultrasonic probe according to claim 1, wherein
the resistance force is generated by a friction between the guide section and the target sample.

8. The ultrasonic probe according to claim 1, wherein
the sensor surface has an elongated shape, and includes a groove portion extending in a longitudinal direction of the sensor surface.

9. The ultrasonic probe according to claim 1, wherein
the ultrasonic sensor section is disposed on the sensor surface so that a scanning direction of the ultrasonic transducer device is oriented parallel to a longitudinal direction of the sensor surface, and
the guide section is configured and arranged to guide a movement of the ultrasonic probe in the longitudinal direction of the sensor surface.

10. An ultrasonic measurement device comprising:
the ultrasonic probe according to claim 1;
a transmitting section configured to perform a process of transmitting ultrasonic waves;
a receiving section configured to perform a process of receiving ultrasonic echoes; and
a processing section configured to perform a process of controlling ultrasonic measurement,
wherein the processing section is configured to generate an ultrasonic panoramic image based on a reception signal from the receiving section.

11. An ultrasonic image device comprising:
the ultrasonic measurement device according to claim 10; and
a display section configured to display image data.
